(19) United States
(12) Patent Application Publication
(10) Pub. No.: US 2010/0069509 A1
(43) Pub. Date: Mar. 18, 2010

(54) NONIONIC EMULSIFIERS FOR EMULSION CONCENTRATES FOR SPONTANEOUS EMULSIFICATION

(75) Inventors: Ulrich Steinbrenner, Neustadt (DE); Astrid Schmidt, Ludwigshafen (DE); Tina Litzel, Hassloch (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: BASF SE, Ludwigshafen (DE)

(21) Appl. No.: 12/517,637

(22) PCT Filed: Dec. 3, 2007
(86) PCT No.: PCT/EP2007/063189

§ 371 Date: Jun. 4, 2009

(30) Foreign Application Priority Data
Dec. 14, 2006 (EP) 06126153.3

(51) Int. Cl. C09K 3/00; C09B 67/00; C09K 3/18; A61K 9/107; C07C 43/02; A01N 25/00; A61K 8/06

(52) U.S. Cl. 514/772; 8/609; 252/70; 252/396; 252/182.12; 568/625; 512/4

(57) ABSTRACT

The present invention relates to a nonionic emulsifier for spontaneous emulsification, containing a chemical compound having the general structure tallow alcohol -n PO-m EO, the average number of hydrocarbon atoms of the tallow alcohol amounting to between 16 and 18, and the iodine count being lower than or equal to 1 g of iodine/100 g of the chemical compound. The average degree of propoxylation is between 1 and 4, and the average degree of ethoxylation is between 2 and 6.

Emulsions are used in technology in many areas:

Thus, for example, corrosion inhibitor emulsions are used as passivating agent for temporarily protecting metallic workpieces against atmospheric corrosion-causing influences. Here, current systems are based on oil concentrates which comprise emulsifiers and corrosion inhibitors, but only little water or no water at all. For the production of oil-in-water emulsions, i.e. for systems which are used in a form diluted with water, it is important that the systems are self-emulsifying.

Likewise, for example, cooling lubricant emulsions are used in the non-cutting or cutting shaping of metallic objects. These have similar compositions to the corrosion inhibitor emulsions and likewise have a corrosion inhibiting effect.

All of these emulsions have the problem that, on account of the emulsifiers used, they have a tendency to form foam. This adversely affects their ability to be used in the various fields of use. It is therefore an object of the present invention to provide a chemical compound which can be used in/as emulsifier, emulsion concentrate and/or emulsion and has a better foaming behavior than the known chemical compounds. Moreover, the chemical compound to be provided should be highly suitable as emulsifier for mineral oils, have high miscibility with oil, good biodegradability, low aquatic toxicity and also high chemical stability.

This object is surprisingly achieved by the chemical compound according to claims 1 to 7, the emulsifier according to claim 8, the emulsifier concentrate according to claims 9 to 11 and the emulsion according to claims 12 to 14. The use according to claim 15 is further provided by the present invention.

A chemical compound of the general structure: tallow fatty alcohol -n PO-m EO, in which the average number of carbon atoms of the tallow fatty alcohol is 16 to 18, the iodine number is less than or equal to 1 g of iodine/100 g of the chemical compound, the alkylene oxide units have an essentially block structure, the average degree of propoxylation is 1 to 4 and the average degree of ethoxylation is 2 to 6, achieves the object according to the invention of providing a low-foaming compound.

This compound is preferred when more than 80% of the alkylene oxide units are arranged in blocks, the average degree of propoxylation is 1 to 3 and the average degree of ethoxylation is 3 to 5. It is particularly preferred when more than 80% of the alkylene oxide units are arranged in blocks, the average degree of propoxylation is 1 to 2 and the average degree of ethoxylation is 4.

As regards the sequence of the alkoxylation units, there are in principle several possibilities: it is possible, starting from the tallow fatty alcohol, for firstly an EO block to follow and then a PO block, likewise it is possible that firstly a PO block and then an EO block follows the tallow fatty alcohol. Gradients or a random distribution are likewise possible. A chemical compound in which the PO block is directly adjacent to the tallow fatty alcohol and the EO block follows it is in accordance with the invention. In this connection, a compound which has “an essentially block structure” is understood as meaning a compound in which, on average, more than 65% of the alkoxylation units are arranged in blocks.

Furthermore, preference is given to chemical compounds as described above in which the weight fraction of EO plus half of the weight fraction of PO is between 35 and 50% of the total weight of the emulsifier, particularly preferably between 40 and 49% and very particularly preferably between 45 and 49%.

The present invention further provides an emulsifier which comprises a chemical compound as described above.

An emulsion concentrate which comprises a chemical compound as described above and/or an emulsifier as described above and a hydrocarbon and/or an ester is likewise further provided by the present invention.

The emulsion concentrate according to the invention can additionally comprise one or more additives selected from the group consisting of water, biocides, corrosion inhibitors, fragrances, pesticides, pharmaceutical agents, buffers, viscosity regulators, antifreezes, antifoams, dyes, complexing agents, salts and coemulsifiers.

Biocides are compounds which kill bacteria. One example of a biocide is glutaraldehyde. The advantage of using biocides is that they counteract the spread of pathogens and increase the shelf-life of the emulsion.

The corrosion inhibitors are, for example, carboxylic acids. These may be straight-chain or branched. Mixtures of different carboxylic acids may be particularly preferred. Caprylic acid, ethylhexanoic acid, isononanoic acid and isodecanoic acid are particularly preferred carboxylic acids. Since corrosion inhibitor emulsions are often neutral to weakly alkaline, it may be advantageous to use the carboxylic acids at least partially in neutralized form, thus as salt. Of suitability for the neutralization are in particular sodium and/or potassium hydroxide solutions, and also alkanolamines. Particular preference is given here to the use of mono- and/or trialkanolamines. The use of dialkanolamines is less preferred due to the danger of the formation of nitrosamines. Nevertheless, dialkanolamines can also be used for the neutralization on their own or together with mono- and/or trialkanolamines.

Fragrances may be individual compounds or mixtures of alcohols, aldehydes, terpenes and/or esters. Examples of fragrances are: lemongrass oil, cochin, dihydromyrcenol, lilial, phenylethyl alcohol, tetrahydrolinalool, hexenol cis-3, lavandin grosso, citral, allyl capronate, citronitriles, benzyl acetate, hexylcinnamaldehyde, citronellol, isoamyl salicylate, isobornyl acetate, terpinyl acetate, linalyl acetate, terpinyl acetate, dihydromyrcenol, agrunitrile, eucalyptus oil, herbaflorat and orange oil. The advantage of using fragrances is that they can provide the composition with a fresh or warning odor and mask troublesome odors.

In the present case, pesticides are understood as meaning all crop protection compositions, as well as compositions for controlling pests. Depending on their target organisms, the pesticides can be further subdivided into: acaricides, algicides, bactericides, fungicides, herbicides, insecticides, molluscicides, nematicides, rodenticides, avicides and virucides.

Pharmaceutical agents comprise all known active ingredients. For the purposes of US patent practice, reference is made expressly to the list of medicaments in Germany, the Rote Liste® 2006 [Red List 2006] and this is incorporated by reference.

Buffers are all compounds which are suitable for essentially keeping the pH of a composition constant during the addition of small amounts of acid or base.

Viscosity regulators serve to adjust the flow properties of liquids.

Antifreezes serve to protect compositions against freezing at low temperatures. Their use enables the composition to be used over a relatively large temperature range. Examples of antifreezes are: glycerol, glycol and ethanol.

Antifoams are formulations with exceptional interface activity which are suitable for suppressing undesired foam formation (e.g. during wastewater purification, papermaking, during the wash cycle in washing machines) or for destroying foam which has already formed. For this purpose, silicone oils with silica particles dispersed therein are widespread—but homogeneous antifoams are also included in the present case.

Dyes may be, besides others: Acid Blue 9, Acid Yellow 3, Acid Yellow 23, Acid Yellow 73, Pigment Yellow 101, Acid Green 1, Acid Green 25. The advantage of using dyes is that they give the composition a certain unmistakable color and thus make them easily distinguishable.

Complexing agents are compounds which are able to bind cations. This can be utilized to reduce the hardness of water and to precipitate out troublesome heavy metal ions. Examples of complexing agents are NTA, EDTA, MGDA and GLDA. The advantage of using these compounds is that many compounds achieve a better effect in soft water; moreover, by reducing the water hardness, the appearance of lime deposits during and after the use of the composition can be reduced or avoided.

Salts can achieve different objects, and the type of salts which can be used according to the invention is therefore very large. Mention may therefore be made, merely by way of example, of the salts of carboxylic acids which, as described above, can be used as corrosion inhibitors.

A further constituent may be coemulsifiers. In this connection, preference is given to an emulsion concentrate in which the coemulsifier(s) is/are selected from the group consisting of ionic surfactants, alcohols and hydrotropes.

Ionic surfactants may be either anionic or cationic surfactants. Examples of anionic surfactants are: carboxylates, sulfonates, sulfo fatty acid methyl esters, sulfates, phosphates. Examples of cationic surfactants are: quaternary ammonium compounds.

Alcohols are compounds which have an OH functionality. Examples are: ethanol, glycol.

Hydrotropes are e.g. salts based on perlagonic acid.

An emulsion which comprises a chemical compound as described above and/or an emulsifier as described above and a hydrocarbon and/or an ester, and water is further provided by the present invention.

An emulsion which additionally comprises one or more additives selected from the group consisting of biocides, corrosion inhibitors, fragrances, pesticides, pharmaceutical agents, buffers, viscosity regulators, antifreezes, antifoams, dyes, complexing agents, salts and coemulsifiers is preferred here. Particular preference is given to an emulsion in which the coemulsifier(s) is/are selected from the group consisting of ionic surfactants, alcohols and hydrotropes.

The use of an emulsion concentrate according to the invention or of an emulsion according to the invention in
metalworking and/or
in the agrochemical sector and/or
in the textile industry and/or
in the leather industry and/or
in the coating industry and/or
in the construction industry and/or
in the plastics processing industry and/or
in the tire industry and/or
in the cleaner industry and/or
in commercial laundry and domestic laundry and/or
in cosmetics and/or
in pharmacy
   is further provided by the present invention.

The invention is illustrated below by examples:

### EXAMPLE 1

### Emulsifier 1 (Comparison)

### Cetyl Oleyl Alcohol×5 EO

By ethoxylating cetyl-oleyl alcohol (iodine number about 60 g of iodine/100 g) with 5 mol equivalents of EO by means of KOH catalysis, cetyl-oleyl alcohol×5 EO was prepared. This emulsifier type is a standard product for the preparation of emulsion concentrates.

### EXAMPLE 2

### Emulsifier 2 (Inventive)

### Tallow Fatty Alcohol×2 PO×4 EO

237 g of tallow fatty alcohol C16C18 with in each case<5% by weight of C14 and C20, iodine number<1 g of iodine/100 g were admixed with 5.0 g of 50% aqueous KOH solution and dewatered for 30 minutes at 120° C. and <20 mbar. Then, at 160° C., 105 g of propylene oxide were gassed in and, after metering had finished, afterreacted for 30 minutes. 160 g of ethylene oxide were then gassed in and aftergassed again for 30 minutes. Finally, the mixture was cooled to 60° C. and neutralized with 5.0 g of 80% lactic acid solution.

### EXAMPLE 3

### Emulsifier 3 (Comparison)

### Tallow Fatty Alcohol×2 PO×7 EO

Tallow fatty alcohol C16C18 was reacted analogously to Example 2 with 2 mol equivalents of PO, but then with 7 mol equivalents of EO.

### EXAMPLE 4

### Foaming Ability in Accordance with EN 12728, 2 g/l of Surfactant, 40° C.

| | |
|---|---|
| Emulsifier 1 | 30 ml |
| Emulsifier 2 | 20 ml |
| Emulsifier 3 | 120 ml |

Emulsifier 2 according to the invention exhibits a lower foaming ability than the comparisons.

### EXAMPLE 5

### Miscibility with Oil

Appearance after storage for 2 months 20% by weight of emulsifier+80% by weight of oil

| Oil, Temperature | Emulsifier 1 | Emulsifier 2 | Emulsifier 3 |
|---|---|---|---|
| SN 150, 23° C. | miscible | miscible | 2 phases |
| SN 150, 50° C. | 2 phases | miscible | miscible |
| SN 500, 23° C. | miscible | miscible | 2 phases |
| SN 500, 50° C. | 2 phases | miscible | miscible |
| Polyalphaolefin, 23° C. | miscible | miscible | 2 phases |
| Polyalphaolefin, 50° C. | 2 phases | miscible | miscible |

Emulsifier 2 according to the invention exhibits better miscibility with oils than the comparison emulsifiers.

### EXAMPLE 6

### Emulsion Stability

The emulsion stability was determined by means of the marker method described in DE 10247086: in two 600 ml beakers, in each case 1% by weight of surfactant was mixed with 69% by weight of water and then 30% by weight of oil—dyed yellow or blue—were added. Then, using a propeller stirrer, a power of about 10 kW/m³ was introduced for 15 minutes.

The resulting emulsions of yellow or blue dyed oil were mixed and then stored at a defined temperature (see table below). At periodic intervals, the emulsions were shaken manually, a sample was taken and the fraction of green drops, formed by coalescence, was determined by means of microscopy and electronic image analysis. The measured green fractions were then plotted against the storage time and fitted by the following function according to the least squares method:

$$Green ( t ) = 100 \% \cdot ( 1 - \frac{2}{2 + r \cdot t} )$$

The fit parameter used is the coalescence rate r. The stability constant S is ultimately obtained from

S=−log(r·month)

The oils used were sunflower oil (56 mm²/s at 25° C.) and paraffin oil (30 mm²/s at 25° C.).

| Oil, Temperature | Emulsifier 1 | Emulsifier 2 | Emulsifier 3 |
|---|---|---|---|
| Sunflower oil, 23° C. | −1.8 | 0.5 | 0.8 |
| Sunflower oil, 70° C. | <−3 | <−3 | −1.3 |
| Paraffin oil, 23° C. | −0.1 | 0.9 | 0.7 |
| Paraffin oil, 70° C. | <−3 | −0.7 | −0.7 |

For paraffin oil, emulsifier 2 according to the invention exhibits a stability better than or comparable to emulsifiers 1 or 3. In the case of the sunflower oil, emulsifier 2 according to the invention is considerably better than emulsifier 1.

### EXAMPLE 7

### Biodegradability

The biodegradability according to OECD 301B is >60% ThCO2 for emulsifier 2 according to the invention.

### EXAMPLE 8

### Aquatic Toxicity According to OECD 202

EC50(Daphnia)=10−100 mg/l

The examples show that the emulsifiers according to the invention are superior to the comparison emulsifiers in some properties essential for the use.

1. A chemical compound of the general structure: tallow fatty alcohol -n PO-m EO, wherein an average number of carbon atoms of the tallow fatty alcohol is 16 to 18; an iodine number is less than or equal to 1 g of iodine/100 g of the chemical compound, an alkylene oxide unit has an essentially block structure; an average degree of propoxylation is 1 to 4; and an average degree of ethoxylation is 2 to 6.

2. The chemical compound according to claim 1, wherein more than 80% of the alkylene oxide units are arranged in blocks; the average degree of propoxylation is from 1 to 3; and the average degree of ethoxylation is from 3 to 5.

3. The chemical compound according to claim 1, wherein more than 80% of the alkylene oxide units are arranged in blocks; the average degree of propoxylation is from 1 to 2; and the average degree of ethoxylation is 4.

4. The chemical compound according to claim 1, wherein the PO block is directly adjacent to the tallow fatty alcohol and the EO block follows the PO block.

5. The chemical compound according to claim 1, wherein the sum of the weight fraction of EO and half of the weight fraction of PO is between 35 and 50% of the total weight of the emulsifier.

6. The chemical compound according to claim 1, wherein the sum of the weight fraction of EO and half of the weight fraction of PO is between 40 and 49% of the total weight of the emulsifier.

7. The chemical compound according to claim 1, wherein the sum of the weight fraction of EO and half of the weight fraction of PO is between 45 and 49% of the total weight of the emulsifier.

8. An emulsifier comprising a chemical compound according to claim 1.

9. An emulsion concentrate comprising
a chemical compound according to claim 1, and
at least one of a hydrocarbon and an ester.

10. The emulsion concentrate according to claim 9, further comprising one or more additives selected from the group consisting of water, a biocide, a corrosion inhibitor, a fragrance, a pesticide, a pharmaceutical agent, a buffer, a viscosity regulator, an antifreeze, an antifoam, a dye, a complexing agent, a salt and a coemulsifier.

11. The emulsion concentrate according to claim 10, wherein the coemulsifier is selected from the group consisting of an ionic surfactant, an alcohol and a hydrotrope.

12. An emulsion comprising
a chemical compound according to claim 1 and
at least of a hydrocarbon and an ester, and water.

13. The emulsion according to claim 12, further comprising one or more additives selected from the group consisting of a biocide, a corrosion inhibitor, a fragrance, a pesticide, a pharmaceutical agent, a buffer, a viscosity regulator, an antifreeze, an antifoam, a dye, a complexing agent, a salt and a coemulsifier.

14. The emulsion according to claim 13, wherein the coemulsifier is selected from the group consisting of an ionic surfactant, an alcohol and a hydrotrope.

15. (canceled)

16. An emulsion comprising an emulsifier according to claim 8 and at least one of a hydrocarbon and an ester and water.

17. An emulsion concentrate comprising an emulsifier according to claim 8 and at least one of a hydrocarbon and an ester.
